(19) United States
(12) Patent Application Publication
(10) Pub. No.: US 2025/0092525 A1
(43) Pub. Date: Mar. 20, 2025

(54) GAS-PHASE REACTOR SYSTEM INCLUDING A GAS DETECTOR

(71) Applicant: ASM IP Holding B.V., Almere (NL)

(72) Inventors: SungBae Kim, Cheonan-si (KR); HakYong Kwon, Hwaseong-si (KR); YoungMin Kim, Hwaseong-si (KR); KiKang Kim, Yongin-si (KR); SeungHwan Lee, Anseong-si (KR)

(21) Appl. No.: 18/967,876

(22) Filed: Dec. 4, 2024

Related U.S. Application Data
(63) Continuation of application No. 17/897,307, filed on Aug. 29, 2022, now Pat. No. 12,195,855.
Application No. 17/897,307 is a continuation of application No. 16/886,186, filed on May 28, 2020, now Pat. No. 11,453,946.
(60) Provisional application No. 62/858,224, filed on Jun. 6, 2019.

(51) Int. Cl. C23C 16/52; C23C 16/44; C23C 16/455; G01M 3/04; H01L 21/67

(52) U.S. Cl. CPC C23C 16/52; C23C 16/4401; C23C 16/4412; C23C 16/45561; H01L 21/67242; H01L 21/67253; G01M 3/04

(57) ABSTRACT

Methods of and systems for performing leak checks of gas-phase reactor systems are disclosed. Exemplary systems include a first exhaust system coupled to a reaction chamber via a first exhaust line, a bypass line coupled to a gas supply unit and to the first exhaust system, a gas detector coupled to the bypass line via a connecting line, a connecting line valve coupled to the connecting line, and a second exhaust system coupled to the connecting line. Methods include using the second exhaust system to exhaust the connecting line to thereby remove residual gas in the connecting line that may otherwise affect the accuracy of the gas detector.

### CROSS-REFERENCE TO RELATED APPLICATIONS

This application is a continuation of, and claims priority to, U.S. patent application Ser. No. 17/897,307 filed Aug. 29, 2022 and titled GAS-PHASE REACTOR SYSTEM INCLUDING A GAS DETECTOR; which is a continuation of U.S. patent application Ser. No. 16/886,186 filed May 28, 2020 and titled GAS-PHASE REACTOR SYSTEM INCLUDING A GAS DETECTOR (now U.S. Pat. No. 11,453,946 issued on Sep. 27, 2022); which claims the benefit of U.S. Provisional Patent Application No. 62/858,224 filed on Jun. 6, 2019, the disclosures of which are incorporated herein in their entirety by reference.

### FIELD OF INVENTION

The present disclosure generally relates to gas-phase methods and systems. More particularly, the disclosure relates to gas-phase systems that include leak detection apparatus and to methods of detecting leaks within the system.

### BACKGROUND OF THE DISCLOSURE

Gas-phase reactors, such as chemical vapor deposition (CVD), plasma-enhanced CVD (PECVD), atomic layer deposition (ALD), and the like can be used for a variety of applications, including cleaning, depositing and etching materials on a substrate surface. For example, gas-phase reactors can be used to clean, deposit and/or etch layers on a substrate to form semiconductor devices, flat panel display devices, photovoltaic devices, microelectromechanical systems (MEMS), and the like.

A typical gas-phase reactor system includes a reactor including a reaction chamber, one or more precursor and/or reactant gas sources fluidly coupled to the reaction chamber, one or more carrier and/or purge gas sources fluidly coupled to the reaction chamber, a gas distribution system to deliver gases (e.g., precursor and/or reactant gas(es) and/or carrier or purge gas(es)) to a surface of a substrate, and an exhaust source fluidly coupled to the reaction chamber.

Many gas-phase reactors include a gas supply unit to supply desired gases to the reaction chamber. A gas supply unit can include one or more sources (or connections to sources), which may be solid, liquid, or gas, at standard room temperature and pressure, valves, including shutoff and/or control valves, lines, heaters, coolers, and the like. The gas supply unit can also include a housing that surrounds the one or more sources, lines, valves, heaters, and/or coolers.

For various reasons, including safety, it may be desirable to detect gas leakage within the gas supply unit. For example, during operation of a gas-phase reactor system, a valve failure or failure of a gas supply block, for example, may lead to leakage of gas, which, in turn, can make it difficult to control the gas flow rate, and can lead to process and/or reactor system failure. Accordingly, it is generally desirable to detect gas leakage within a gas supply unit as soon as possible.

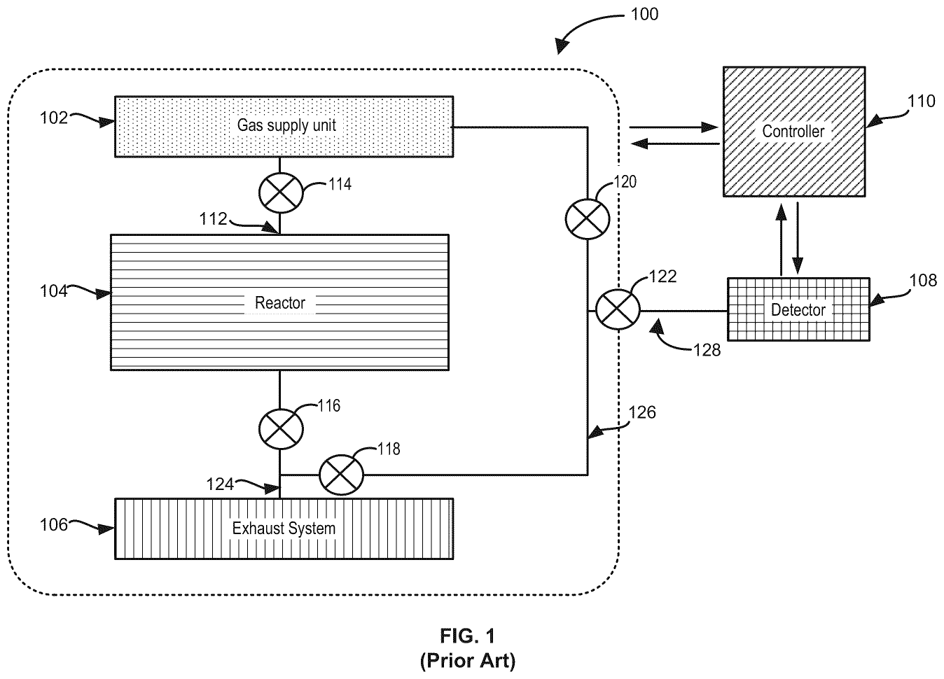

FIG. 1 illustrates a gas-phase reactor system 100 that includes a gas supply unit 102, a reactor 104, an exhaust system 106, a gas detector 108, and a controller 110. System 100 also includes a gas inlet 112, valves 114-122, and an exhaust path 124.

In the illustrated example, gas supply unit 102 and reactor 104 are connected via gas inlet 112. Process gas is supplied to reactor 104 through a gas inlet valve 114 and gas inlet 112. Gas inlet valve 114 can be a part of gas supply unit 102 and gas inlet 112 can include a gas supply apparatus, such as a showerhead or the like. Gas from reactor 104 is exhausted to exhaust system 106 via exhaust path 124. Exhaust system 106 can include, for example, a dry pump, a scrubber or the like. Exhaust path 124 can include exhaust valve 116. Exhaust valve 116 can function to control a pressure in the reactor 104 by being equipped with a pressure control device, e.g., a butterfly wing plate, and it may be controlled by an exhaust valve control unit (not shown) that communicates with a pressure gauge (not shown) installed at reactor 104.

System 100 also includes a divert or bypass path 126. Divert path 126 is connected to gas supply unit 102 and exhaust path 124, and bypasses gas inlet 112, reactor 104 and valve 116. Divert path 126 is especially useful in ALD-type processes to facilitate keeping a process pressure in reactor 104 constant during a process, because divert path 126 can be used to switch a gas flow direction to divert path 126 from reactor 104 by adjusting valve movements, without increasing or decreasing the gas flow rate. By keeping the process pressure constant, pressure fluctuation in the gas supply line and reactor 104 may be minimized and the process may be more stable.

In the illustrated example, divert path 126 includes a first divert valve 120, a second divert valve 122 and a third divert valve 118. When gas is supplied to reactor 104, first divert valve 120 and a third divert valve 118 are closed. When gas is supplied to divert path 126, first divert valve 120 and third divert valve 122 are open.

Gas detector 108 is fluidly coupled to divert path 126 to check for gas leakage within gas supply unit 102. For example, when a valve or a part of an integrated gas supply system block of the gas supply unit fails and outer gas leaks into the gas supply unit 102 through a failed portion of the integrated gas supply system block, gas detector 108 may detect the leaking gas and send a signal to controller 110, and controller 110 can cause stoppage of the operation of a portion of the system 100 (dotted line area of FIG. 1).

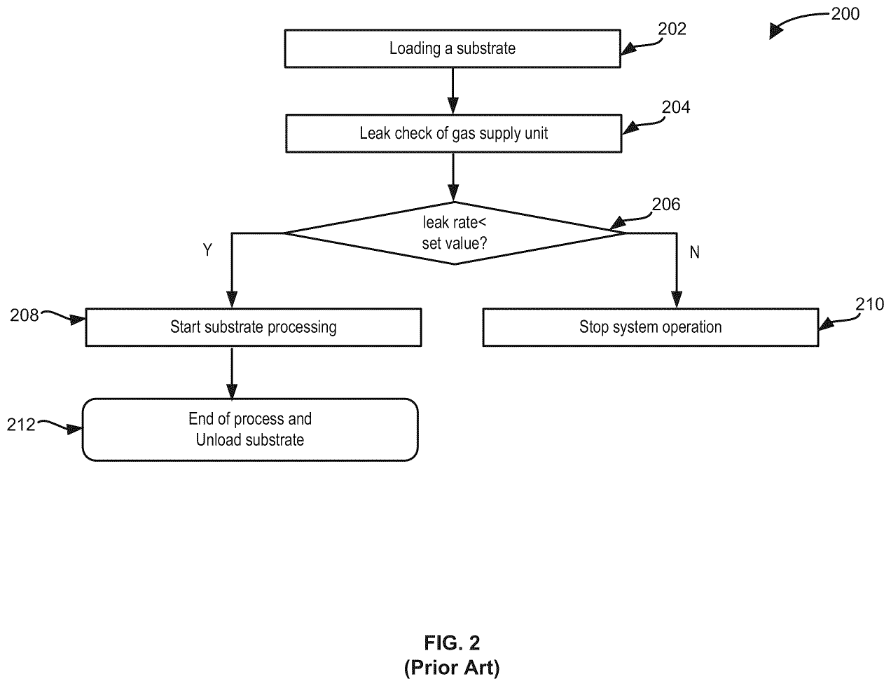

FIG. 2 illustrates a leak check method 200 of a gas-phase reactor system, such as gas-phase reactor system 100. Method 200 includes the steps of loading a substrate within a reactor (step 202), leak checking the gas supply unit (step 204), determining whether a leak rate is greater than a predetermined value (step 206), start substrate processing (step 208), stop system operation (step 210), and end process and unload substrate (step 212).

During step 202, a substrate is loaded to the reactor (e.g., reactor 104). The substrate may be mounted on, for example, a susceptor or a heating block.

During steps 204 and 206, a leak check of the gas supply unit 102 is carried out. During step 204, all valves of the gas supply unit 102 are open, and the gas inlet valve 114 and foremost valves (not illustrated) of gas supply unit 102, through which gases, such as precursors, reactants and other process gases flow into gas supply unit 102 from a gas reservoir or vessel (not shown) are closed. Instead, first divert valve 120, second divert valve 122 and third divert valve 118 are open. In this case, all portions of gas supply unit 102 are in fluid communication with divert path 126 and gas detector 108, without being in open fluid communication with gas inlet 112 and reactor 104 and gas reservoirs or vessels external to gas supply unit 102. During step 204, gas detector 108 detects any residual gas exhausted from gas supply unit 102, flowing to divert path 126. During step 206, if the residual gas contains outer gas, such as N₂ or O₂, originated from the atmosphere, and as a result, the leak rate of the gas is over the set value, gas detector 108 can send a signal to controller 110. If a leak is detected, controller 110 can be configured to cause stoppage of the operation system 100 (step 210). Steps 204 and 206 can be performed during a preprocess step of the substrate, such as a preheating step.

Based on the detection results during step 206, a process may be performed (step 208). The process may be or include film deposition, etching, ashing, cleaning, or the like. If the detection results exceed the set value, the substrate processing system may stop the operation (step 210). In other embodiments, the detection results may be synchronized with an interlock system. In this case, if the detection result is over the set value, the interlock system stops the operation.

At step 212, when the process is completed, a substrate is unloaded from the reactor and the next substrate is loaded and steps 202-212 are repeated. In other words, the leak detection of the gas supply unit 102 is performed repeatedly after a substrate is loaded within a reaction chamber and before processing the substrate from the reaction chamber.

Leak detection of gas supply unit 102 using system 100 and method 200 may exhibit low accuracy in detecting outer gas due to trapped gas in an area 128. Area 128 may be a gas pipe connecting divert path 126 and gas detector 108. Area 128 desirably includes minimal residual gas or trapped gas in it for accurate leak detection of gas supply unit 102 after completing step 204 and step 206. But, due to the subsequent substrate processing step 208 right after the leak detecting steps 204 and 206, second divert valve 122 is closed to protect gas detector 108 from process gas flowing into divert path 126, thereby trapping gas in the area 128. The trapped gas in area 128 may obstruct the accurate and precise leak detection during step 204 and step 206 before processing the next substrate. In another case, gas from the gas supply unit may be accumulated in area 128 during steps 204 and 206, and the accumulated gas may make it difficult to detect the outer gas accurately. Accordingly, improved systems and methods for detecting leaks in gas-phase reactor systems are desired.

Any discussion of problems and solutions set forth in this section has been included in this disclosure solely for the purposes of providing a context for the present disclosure, and should not be taken as an admission that any or all of the discussion was known at the time the invention was made.

### SUMMARY OF THE DISCLOSURE

Various embodiments of the present disclosure relate to gas-phase reactor systems and methods. While the ways in which various embodiments of the present disclosure address drawbacks of prior methods and systems are discussed in more detail below, in general, exemplary embodiments of the disclosure provide improved systems and methods for detecting gas leaks within a gas-phase reactor system.

In accordance with at least one embodiment of the disclosure, a gas-phase reactor system includes a reactor comprising a reaction chamber; a gas supply unit coupled to the reaction chamber via a gas supply line; a first exhaust system coupled to the reaction chamber via a first exhaust line; a bypass line coupled to the gas supply unit and to the first exhaust system; a gas detector coupled to the bypass line via a connecting line; a connecting line valve coupled to the connecting line; and a second exhaust system coupled to the connecting line. In accordance with exemplary aspects of these embodiments, the bypass line is coupled to the gas supply line. The bypass line can also be coupled to the first exhaust line. In accordance with further aspects, the gas-phase reactor system includes a second exhaust line coupled to the connecting line. A second exhaust line valve can be between the connecting line and the second exhaust system. Further, the second exhaust system can be coupled to a chamber, such as a platform chamber or an outer chamber. The gas detector can detect a flowrate and/or a composition (e.g., nitrogen and/or oxygen content) of a gas. The gas-phase reactor system can also include a controller configured to: cause the gas-phase reactor system to perform a leak test after a substrate has been loaded into the reaction chamber and before the substrate is removed from the reaction chamber, cause the gas-phase reactor system to perform a leak test while heating a substrate to a desired process temperature, perform a leak test during a process cycle, stop flow of gas to the reaction chamber when the gas detector detects a flow rate of gas above a predetermined value, stop operation of the gas-phase reactor system when the gas detector detects a flow rate of gas above a predetermined value, exhaust the connecting line during a substrate process within the reaction chamber, and/or exhaust the connecting line by closing the connecting line valve and opening the second exhaust line valve.

In accordance with at least one other embodiment of the disclosure, a method of using a gas-phase reactor system includes the steps of providing a gas-phase reactor system, such as providing a gas-phase reactor system described herein, exhausting the connecting line, and, using the gas detector, analyzing gas exhausted from the gas-phase reactor system. The gas can be exhausted from the gas supply unit. The method can further include a step of closing a second exhaust line valve between the gas detector and the second exhaust system after the step of exhausting the connecting line and prior to the step of analyzing gas. Additionally or alternatively, the method can include a step of exhausting the bypass line during the step of analyzing. Exemplary methods can include a step of loading a substrate within the reaction chamber, wherein the step of analyzing is performed after the step of loading and before a step of unloading the substrate from within the reaction chamber. Alternatively, the method can include a step of loading a substrate within the reaction chamber, wherein the step of analyzing is performed before the step of loading. During the step of analyzing, gas can be exhausted to the second exhaust system. The gas detector can be used to detect a composition and/or flowrate of a gas. For example, the gas detector can be used to determine whether a gas flow rate is above a predetermined level. In this case, the method can include if the gas flow rate is above the predetermined level, stopping flow of gas to the reaction chamber, if the gas flow rate is above the predetermined level, stopping operation of the gas-phase reactor system, and/or if the gas flow rate is above the predetermined level, engaging an interlock system. The step of analyzing can be performed while heating a substrate to a desired process temperature, during a process cycle, and/or after a substrate has been loaded into the reaction chamber and before the substrate is removed from the reaction chamber.

These and other embodiments will become readily apparent to those skilled in the art from the following detailed description of certain embodiments having reference to the attached figures; the invention not being limited to any particular embodiment(s) disclosed.

BRIEF DESCRIPTION OF THE DRAWING FIGURES

A more complete understanding of exemplary embodiments of the present disclosure can be derived by referring to the detailed description and claims when considered in connection with the following illustrative figures.

FIG. 1 illustrates a gas-phase reactor system of the prior art.

FIG. 2 illustrates a leak detection method of the prior art.

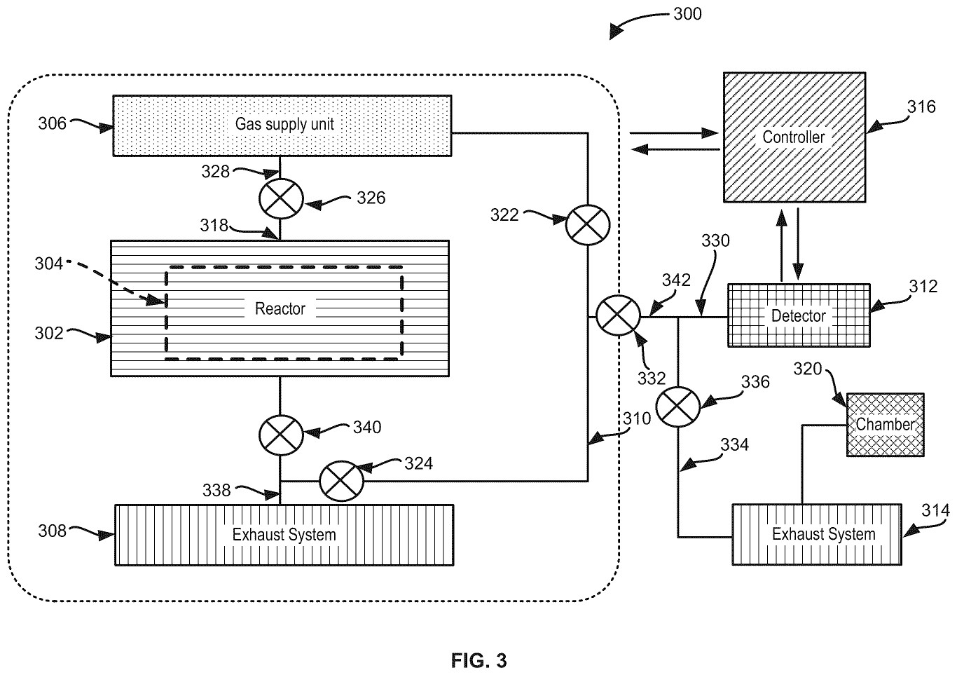

FIGS. 3-7 and 10 illustrate a gas-phase reactor system in accordance with at least one embodiment of the disclosure.

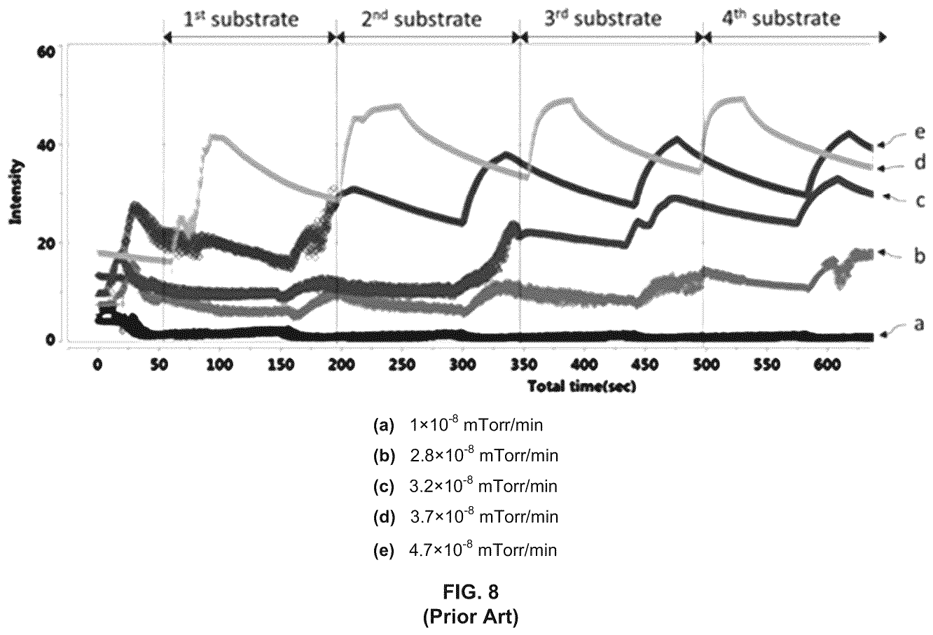

FIG. 8 illustrates leak check results using a system known in the art.

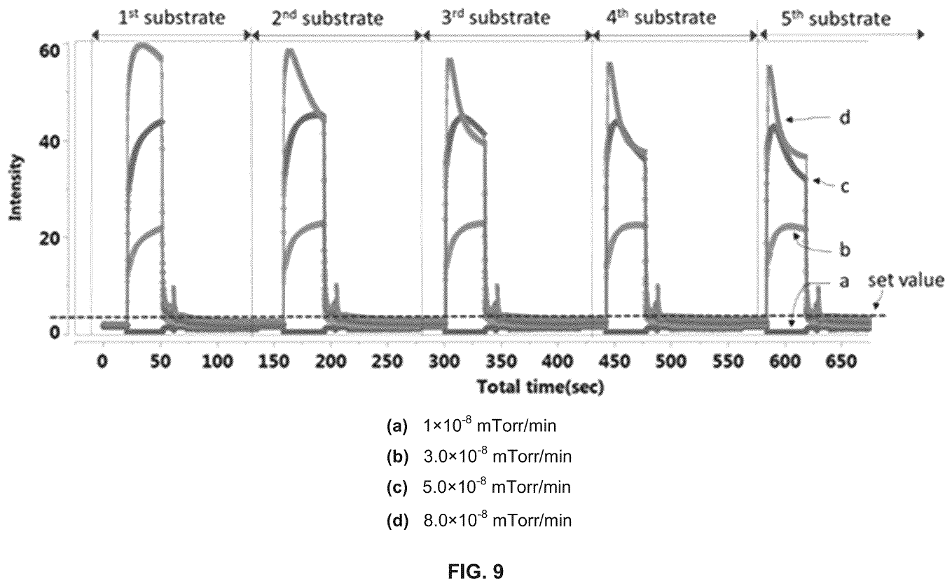

FIG. 9 illustrates leak check results using a system or method in accordance with at least one embodiment of the disclosure.

It will be appreciated that elements in the figures are illustrated for simplicity and clarity and have not necessarily been drawn to scale. For example, the dimensions of some of the elements in the figures may be exaggerated relative to other elements to help improve understanding of illustrated embodiments of the present disclosure.

### DETAILED DESCRIPTION OF EXEMPLARY EMBODIMENTS

Although certain embodiments and examples are disclosed below, it will be understood by those in the art that the invention extends beyond the specifically disclosed embodiments and/or uses of the invention and obvious modifications and equivalents thereof. Thus, it is intended that the scope of the invention disclosed should not be limited by the particular disclosed embodiments described below.

The present disclosure generally relates to gas-phase reactor systems and methods capable of determining a leak. As set forth in more detail below, exemplary systems and methods described herein can be used to more accurately determine a composition, flow rate, and/or amount of gas leaking from one or more areas or sections of a gas-phase reactor system. Further, the systems and methods described herein may be more efficient in processing substrates and performing leak checks, compared to traditional methods and systems.

In this disclosure, “gas” can include material that is a gas at room temperature and pressure, a vaporized solid and/or a vaporized liquid, and may be constituted by a single gas or a mixture of gases, depending on the context. A gas other than the process gas, i.e., a gas introduced without passing through a gas distribution assembly, such as a showerhead, other gas distribution device, or the like, may be used for, e.g., sealing the reaction space, which includes a seal gas, such as a rare gas. A gas can be a reactant or precursor that takes part in a reaction within a reaction chamber and/or include ambient gas, such as air.

In this disclosure, “line” can refer to a conduit, such as a tube, through which gas flows. A line can include one or more valves, branches, or the like. Exemplary lines as described herein can be formed of stainless steel.

In this disclosure, any two numbers of a variable can constitute a workable range of the variable as the workable range can be determined based on routine work, and any ranges indicated may include or exclude the endpoints. Additionally, any values of variables indicated (regardless of whether they are indicated with “about” or not) may refer to precise values or approximate values and include equivalents, and may refer to average, median, representative, majority, etc. in some embodiments. Further, in this disclosure, the terms “constituted by” and “having” refer independently to “typically or broadly comprising,” “comprising,” “consisting essentially of,” or “consisting of” in some embodiments. In this disclosure, any defined meanings do not necessarily exclude ordinary and customary meanings in some embodiments.

In this disclosure, “continuously” can refer to one or more of without breaking a vacuum, without interruption as a timeline, without any material intervening step, without changing treatment conditions, immediately thereafter, as a next step, or without an intervening discrete physical or chemical structure between two structures other than the two structures in some embodiments.

Turning again to the figures, FIG. 3 illustrates a gas-phase reactor system 300 in accordance with exemplary embodiments of the disclosure. Gas-phase reactor system 300 includes a reactor 302 comprising a reaction chamber 304, a gas supply unit 306, a first exhaust system 308, a bypass line 310 coupled to gas supply unit 306 and to the first exhaust system 308, a gas detector 312, and a second exhaust system 314. Gas-phase reactor system 300 can also include a controller 316 to control various portions or devices of gas-phase reactor system 300.

Reactor 302 can include any suitable gas-phase reactor. By way of examples, reactor 302 can be configured as a chemical vapor deposition reactor, an atomic layer deposition reactor, an etch reactor, a clean reactor, an epitaxial reactor, or the like. In some cases, reactor 302 can include a direct plasma configuration and/or gas-phase reactor system 300 can include a remote plasma unit coupled to reactor 302. Reactor 302 includes a gas inlet 318 to receive gas from gas supply unit 306.

Gas supply unit 306 supplies one or more process gases, such as one or more precursors and/or one or more reactants to reaction chamber 304 through gas inlet 318. Gas supply unit 306 can also provide a carrier and/or inert gas to the reaction chamber through gas inlet 318. Gas supply unit 306 can include an integrated gas supply block. The integrated gas block system is a block-typed or Lego-typed gas supply system, so as to make the whole gas supply path from the foremost valve to the hindmost valve simple, compact and short, and reduce the blind spots between gas supply path and a valve, compared to conventional plumbing-typed gas supply systems. So the gas supply or switching between gases may be faster in the integrated gas block system than in the conventional plumbing system.

First exhaust system 308 and second exhaust system 314 can include any suitable device to exhaust a line and/or reaction chamber. By way of examples, first exhaust system 308 and/or second exhaust system 314 can be or include a dry pump, a scrubber, a turbomolecular pump, or the like. As illustrated in FIG. 3, second exhaust system 314 can be coupled to another chamber 320, such as a platform chamber for transferring substrates between a reactor and a cooler or load-lock, or an outer chamber with a rotation arm encompassing multiple reactors in a multi-reactor chamber.

A line 338 can connect reactor 302 to first exhaust system 308. As illustrated, line 338 can include a valve 340, which can be coupled to and controlled by controller 316.

Bypass line 310 is coupled to gas supply unit 306 and to first exhaust system 308. Bypass line 310 includes a first bypass line valve 322 between gas supply unit 306 and first exhaust system 308. Bypass line 310 can additionally or alternatively include a second bypass line valve 324. First and second bypass line valves 322 and 324 can include any suitable type valve, such as a pneumatic valve. First and second bypass line valves 322 and/or 324 can be coupled to and controlled by controller 316.

Gas detector 312 can detect or measure a flowrate and/or composition of a gas. By way of examples, gas detector 312 can be or include, for example, SPOES (Self-Plasma Optical Emission Spectroscopy) which decomposes gas, analyzes and detects a type of gas. For example, since nitrogen takes up about 70% of the atmosphere, gas detector 312 may be configured to detect nitrogen in a gas. In some cases, if the nitrogen is detected by gas detector 312, and the detected nitrogen is over the set value, it may be determined that an outer gas (e.g., from an environment surrounding gas supply unit 306) has leaked into gas supply unit 306 and/or elsewhere within gas-phase reactor system 300. Additionally or alternatively, gas detector 312 can include a flow meter and/or a mass flow meter to determine an amount or a flowrate of a leak.

A connecting line 330 can connect bypass line 310 to gas detector 312. Connecting line 330 can include a connecting line valve 332, which can be a pneumatic valve and which can be coupled to controller 316.

Controller 316 can be any suitable controller that can cause various steps or functions as described herein to be performed. In accordance with various examples of the disclosure, controller 316 receives signals from gas detector 312 that can indicate a composition, flowrate, and/or amount of gas. As discussed in more detail below, controller 316 can be configured to cause one or more of: cause the gas-phase reactor system to perform a leak test after a substrate has been loaded into the reaction chamber and before the substrate is removed from the reaction chamber, cause the gas-phase reactor system to perform a leak test while heating a substrate to a desired process temperature, perform a leak test during a process cycle, stop flow of gas to the reaction chamber when the gas detector detects a flow rate of gas above a predetermined value, stop operation of the gas-phase reactor system when the gas detector detects a flow rate of gas above a predetermined value, exhaust the connecting line during a substrate process within the reaction chamber, and exhaust the connecting line by closing connecting line valve 332 and opening a second exhaust line valve 336 based on one or more signals received from gas detector 312.

Gas supply unit 306 and reactor 302 are connected via gas inlet 318. Process gas can be supplied to reactor 302 through a gas inlet valve 326 in a gas inlet line 328. Although separately illustrated, gas inlet valve 326 can be a part of gas supply unit 306. Gas inlet 318 can include a gas supply apparatus, such as showerhead or the like.

As illustrated, gas-phase reactor system 300 can include a second exhaust line 334 coupled to connecting line 330. Second exhaust line 334 can also be coupled to second exhaust system 314. Second exhaust line 334 can include second exhaust line valve 336, which can be a pneumatic valve or the same or similar to a check valve through which gas flows forward, not flowing back, and which can be coupled to and controlled by controller 316.

As set forth in more detail below, use of gas-phase reactor system 300 has several advantages over use of conventional gas-phase reactor systems. For example, while processing a substrate using gas-phase reactor system 300, during a substrate processing (e.g., deposition, etch, or clean step), connecting line valve 332 can be closed (e.g., using controller 316) and second exhaust line valve 336 can be open (e.g., opened using controller 316), such that residual gas trapped or accumulated gas in an area (i.e., blind spot) 342 may be exhausted to second exhaust system 314 through second exhaust line 334. In other words, by adopting this system, any gas that would otherwise be trapped in connecting line 330 can be mitigated, thereby improving the accuracy of measurements performed using gas detector 312. Further, process gas can include compounds that are generated by the reaction between process gases in areas between reaction chamber 304 and first exhaust system 308; these compounds can be deposited and stuck in first exhaust system 308. This may make switching between exhaust of gas in bypass line 310, coupled to gas supply unit 306 and to first exhaust system 308, and line 338 coupled between reaction chamber 304 and first exhaust system 308, to first exhaust system 308 not be smooth. However, using system 300, second exhaust line 334 and the second exhaust system 314 can be used mitigate abrupt transition to first exhaust system 308 and thereby provide for more accurate analysis and detection of, for example, outer gas leaked into the gas supply unit 306 and other gas leaks within gas-phase reactor system 300. Further, outgassing effect from the chemical compounds deposited on the inside wall of line 338 can obstruct the smooth exhaust from bypass line 310. This effect is especially severe in processes that generate a lot of by-products such as powder, for example, SiN process using DCS(dichlorosilane) and NH₃ as process gas, and lower the accuracy of the analysis of leaked gas into the gas supply unit 306 because of the residual gas in the bypass line 310. So it's necessary to remove residual gas in the bypass line 310 for accurate analysis of leaked gas into the gas supply unit 306.

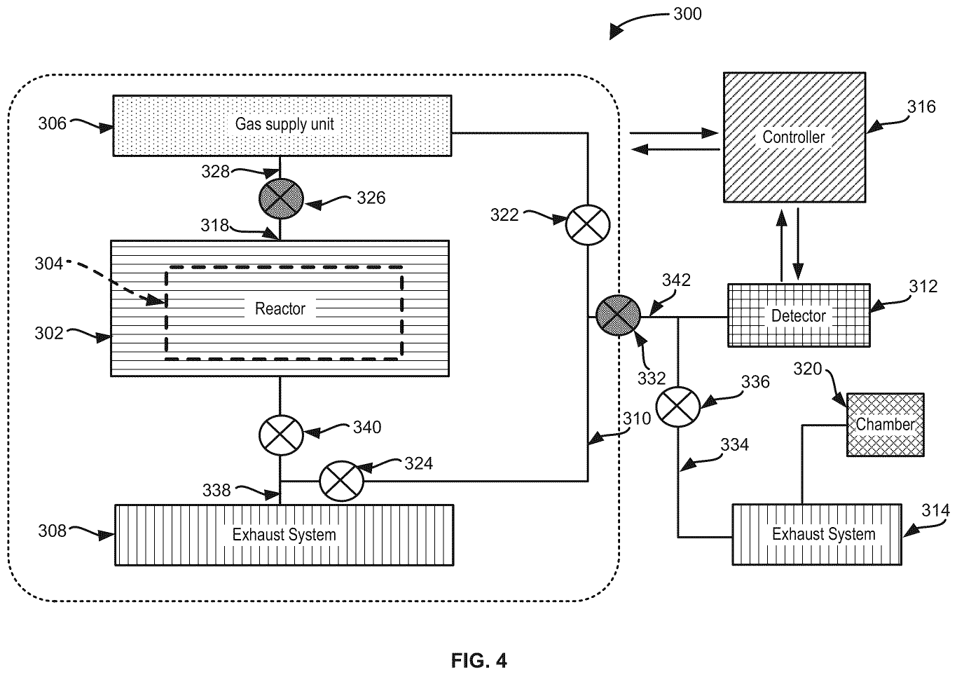

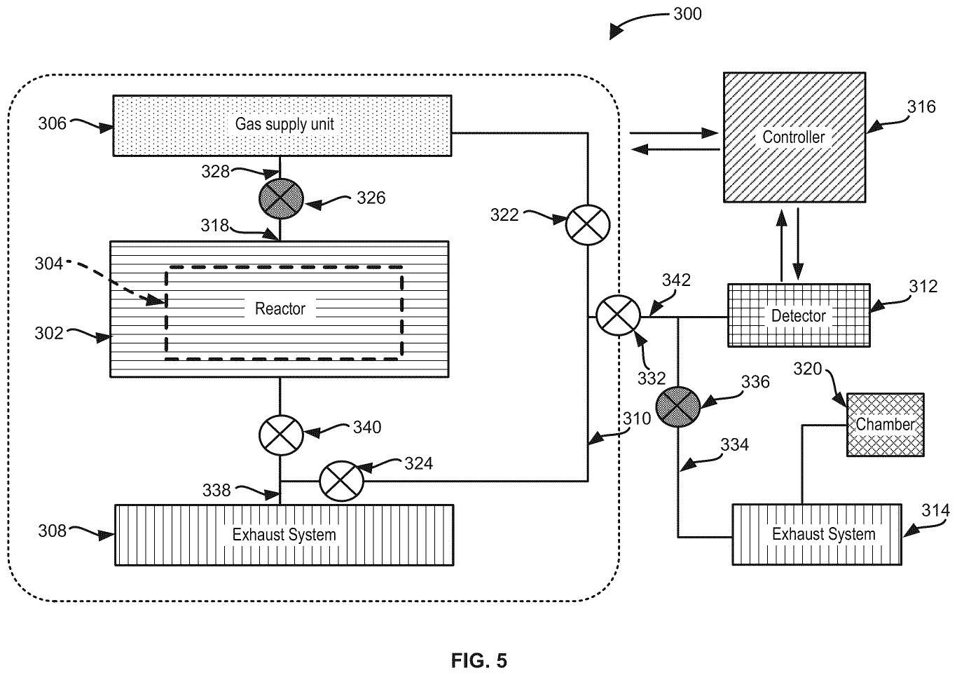

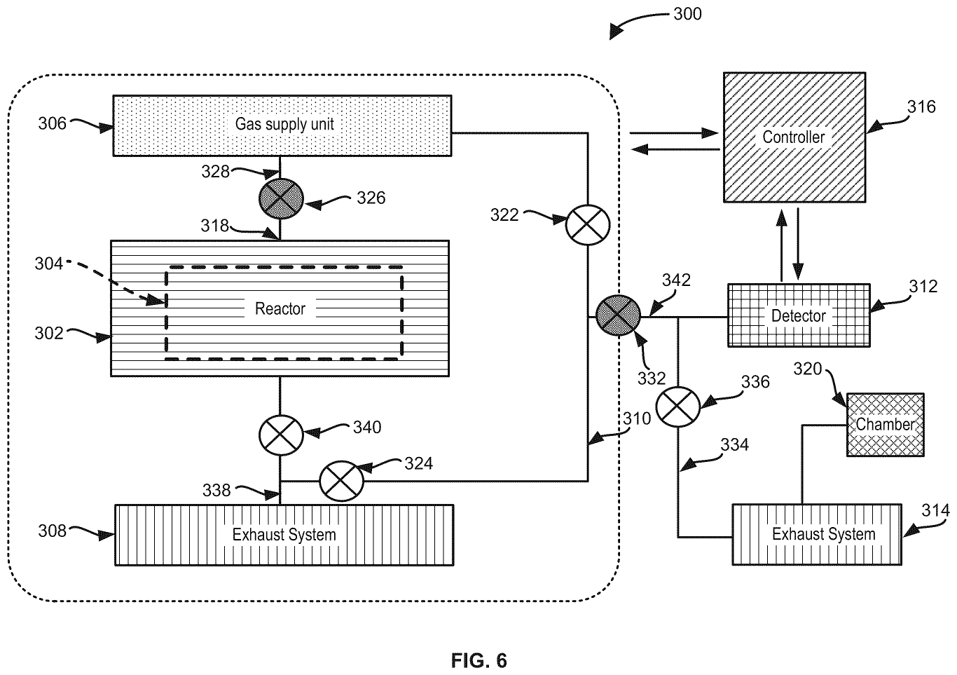

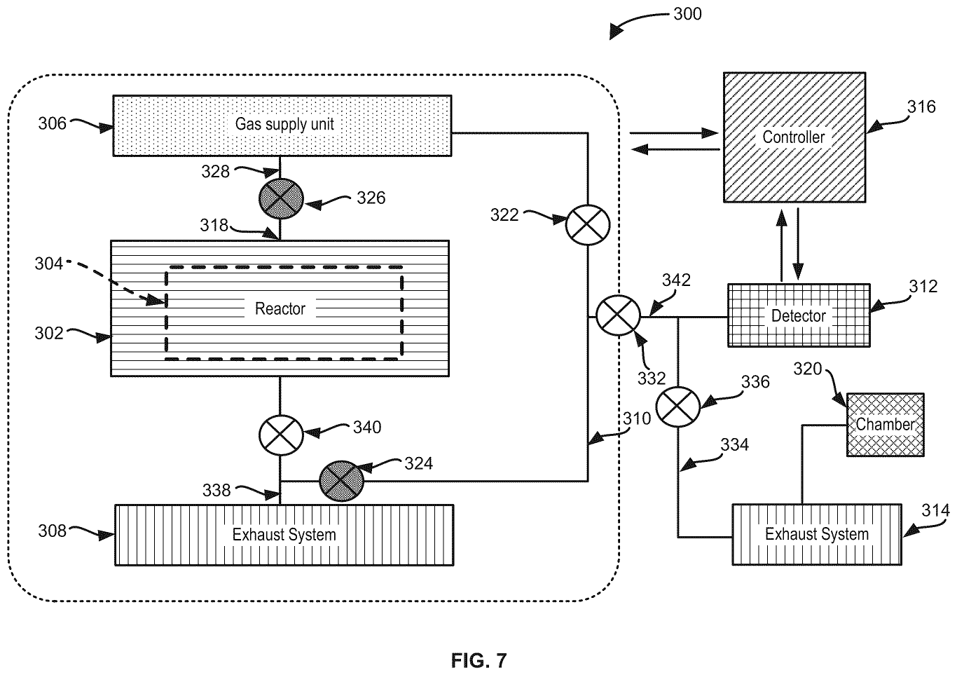

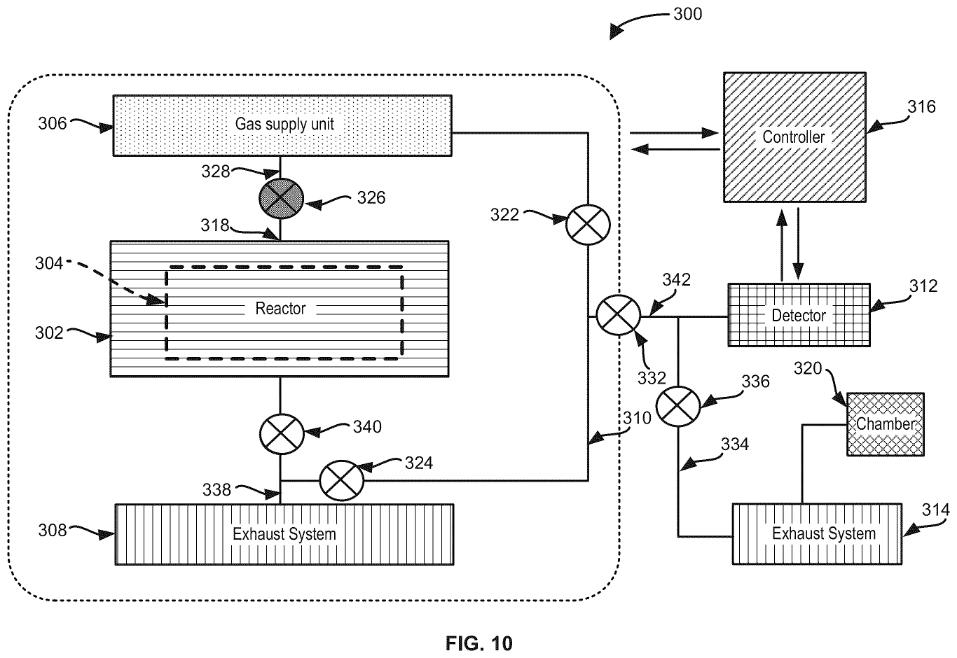

FIGS. 4-7 and 10 illustrate gas-phase reactor system 300 during processing in accordance with additional embodiments of the disclosure. FIGS. 4 and 5 illustrate gas-phase reactor system 300 while performing a leak check after loading a substrate within a reaction chamber (e.g., reaction chamber 304) and prior to processing the substrate (e.g., prior to introducing reaction gases into reaction chamber 304). FIGS. 6 and 7 illustrate another example of gas-phase reactor system 300 while performing a leak check step after loading a substrate and before processing the substrate. And, FIG. 10 illustrates yet another example of gas-phase reactor system 300 while performing a leak check step.

As illustrated in FIG. 4, after a substrate is loaded within reaction chamber 304, first bypass line valve 322, second bypass line valve 324 and second exhaust line valve 336 are initially open or can be opened using controller 316. Gas inlet valve 326 is initially closed or can be closed using controller 316. Connecting line valve 332 is closed for certain period of time, e.g., less than 10 seconds, e.g., using controller 316, so as to remove any potential residual gas area 342 between a connecting line valve 332 and a gas detector 312.

Next, as illustrated in FIG. 5, first bypass line valve 322, connecting line valve 332 and second bypass line valve 324 are open (e.g., by opening using controller 316). Gas inlet valve 326 is closed (e.g., using controller 316). Second exhaust line valve 336 is closed for certain period of time, e.g., less than 5 seconds—e.g., using controller 316. Next, gas detector 312 starts detecting and analyzing gas exhausted from the gas supply unit 306 and/or elsewhere in gas-phase reactor system 300.

As illustrated in FIG. 6, in accordance with another embodiment of the disclosure, after a substrate is loaded within reaction chamber 304, first bypass line valve 322, second bypass line valve 324 and second exhaust line valve 336 are open or can be opened using, for example, controller 316. Gas inlet valve 326 is closed or can be closed using, for example, controller 316. Connecting line valve 332 is closed for certain period of time, e.g., less than 10 or 5 seconds, using, e.g., controller 316, so as to remove any potential residual gas from the area 342 between connecting line valve 332 and gas detector 312.

Next, as illustrated in FIG. 7, first bypass line valve 322, connecting line valve 332 and second exhaust line valve 336 are open or are opened—e.g., using controller 316. Gas inlet valve 326 is closed—e.g., using controller 316. Second bypass line valve 324 is closed, e.g., using controller 316, for certain period of time, e.g., less than 10 or 5 seconds. Gas detector 312 then starts detecting and analyzing gas exhausted from the gas supply unit 306. In this embodiment, gas exhausted from gas supply unit 306 is exhausted to second exhaust system 314, so that any blocking effect from first exhaust system 308 and/or a first exhaust path (e.g., line 338) can be avoided. This procedure may be particularly useful in processes capable of producing a lot of byproducts, such as powder, e.g., silicon nitride deposition processes that use DCS and NH₃ as process gases.

In accordance with illustrative examples, during processing of a substrate, connecting line valve 332 is closed and second exhaust line valve 336 is opened (e.g., using controller 316) to prevent or mitigate any residual gas from being detected during processing a substrate.

FIG. 10 illustrates another example, in which bypass line valve 322, connecting line valve 332, second bypass line valve 324, and second exhaust line valve 336 are open in a leak check step. In this case, gas can be evacuated from line 342 as described above by closing connecting line valve 332 certain period of time, e.g., less than 10 or 5 seconds, using, e.g., controller 316, so as to remove any potential residual gas from the area 342 between connecting line valve 332 and gas detector 312. Then, bypass line valve 322, connecting line valve 332, second bypass line valve 324 and second exhaust line valve 336 are open or can be opened—e.g., using controller 316—during a leak check, such that gas is exhausted to both first exhaust system 308 and second exhaust system 314 during the leak check. Valve 340 can be open during this step. This example can be performed before or after processing a substrate.

FIG. 8 illustrates leak check results using the gas-phase reactor system illustrated in FIG. 1. FIG. 8 illustrates consolidated graphs, showing nitrogen intensity per each leak rate test of gas supply unit 102 when multiple substrates (four in the illustrative example) were processed successively. In FIG. 8, the Y-axis is the intensity of nitrogen detected by a gas detector (e.g., gas detector 108) at each leak rate test. The X-axis is a substrate process time which includes a leak detecting step. It took about 150 seconds for each substrate to be processed.

As illustrated in FIG. 8, the more the leak rate increases, the greater the nitrogen intensity. But, the nitrogen intensity is not uniform and gradually increases as multiple substrates are successively processed. In addition, the nitrogen intensity is detected during the entire substrate processing time, and a leak detecting step is not distinct from a substrate processing step. This is because the gas detector (e.g., gas detector 108) keeps detecting the residual gas in the blind spot area 128 in FIG. 1 during substrate processing step—even though valve 122 is closed. The trapped residual gas can accumulate in the blind spot area and can affect the detection results of the following substrate. For example, some of the nitrogen intensity of the second substrate may come from that of residual gas trapped in the blind spot area during substrate processing of the first substrate. So the leak detection and analysis of the gas supply unit is not accurate and is unreliable.

In contrast, FIG. 9 illustrates the leak check results using gas-phase reactor system 300 in accordance with examples of the disclosure. Contrary to the results illustrated in FIG. 8, FIG. 9 illustrates a leak detecting step is clearly distinct from a substrate processing step. A nitrogen intensity is uniform regardless of the number of processed substrates. This is thought to be because residual gas in the blind spot area 342 is exhausted to the second exhaust system 314 through a second divert path before the leak detecting step begins.

Therefore, according to examples of the disclosure, a set (e.g., intensity, flowrate, or the like) value may be made. If a measured value (e.g., intensity, flowrate, or the like) is over the set value, an interlock system may be used to stop the operation of system 300. As mentioned above, during processing a substrate at the substrate processing step, connecting line valve 332 is closed and second exhaust line valve 336 is open to prevent any residual gas from being detected during processing a substrate.

The introduction of second exhaust line 334, as described herein, removes residual gas trapped between a second divert valve and a gas detector, and provides more accurate, more reliable leak detection and analysis results of the gas supply unit. In addition, methods as described herein that provide a leak check of the gas supply unit before processing a substrate may prevent potential damage to the substrate by being synchronized with an interlock system. Further, a detection may be performed during a preheating step of the substrate, so the detection does not affect the through-put.

The example embodiments of the disclosure described above do not limit the scope of the invention, since these embodiments are merely examples of the embodiments of the invention. Any equivalent embodiments are intended to be within the scope of this invention. Indeed, various modifications of the disclosure, in addition to those shown and described herein, such as alternative useful combinations of the elements described, may become apparent to those skilled in the art from the description. Such modifications and embodiments are also intended to fall within the scope of the appended claims.

We claim:

1. A gas-phase reactor system comprising:
a reactor comprising a reaction chamber;
a gas supply unit coupled to the reaction chamber via a gas supply line;
a first exhaust system coupled to the reaction chamber via a first exhaust line;
a bypass line coupled to the gas supply unit and to the first exhaust system;
a gas detector coupled to the bypass line via a connecting line;
a second exhaust line coupled to the connecting line;
a connecting line valve coupled to the connecting line; and
a second exhaust line valve between the connecting line and a second exhaust system.

2. The gas-phase reactor system of claim 1, wherein the bypass line is coupled to the gas supply line.

3. The gas-phase reactor system of claim 1, wherein the bypass line is coupled to the first exhaust line.

4. The gas-phase reactor system of claim 1, wherein the second exhaust system is coupled to a chamber.

5. The gas-phase reactor system of claim 1, further comprising a first bypass line valve between the gas supply unit and the first exhaust system.

6. The gas-phase reactor system of claim 5, further comprising a second bypass line valve between the gas supply unit and the first exhaust system.

7. The gas-phase reactor system of claim 1, wherein the gas detector can detect a flow rate of a gas.

8. The gas-phase reactor system of claim 1, wherein the gas detector can detect a composition of a gas.

9. The gas-phase reactor system of claim 1, further comprising a controller configured to: cause the gas-phase reactor system to perform a leak test.

10. The gas-phase reactor system of claim 1, further comprising a controller configured to: cause the gas-phase reactor system to perform a leak test after a substrate has been loaded into the reaction chamber and before the substrate is removed from the reaction chamber.

11. The gas-phase reactor system of claim 1, further comprising a controller configured to:
perform a leak test during a process cycle.

12. The gas-phase reactor system of claim 1, further comprising a controller configured to: stop flow of gas to the reaction chamber when the gas detector detects a flow rate of gas above a predetermined value.

13. The gas-phase reactor system of claim 1, further comprising a controller configured to: stop operation of the gas-phase reactor system when the gas detector detects a flow rate of gas above a predetermined value.

14. A gas-phase reactor system comprising:
a reactor comprising a reaction chamber;
a gas supply unit coupled to the reaction chamber via a gas supply line;
a first exhaust system coupled to the reaction chamber via a first exhaust line;
a bypass line coupled to the gas supply unit and to the first exhaust system;
a gas detector coupled to the bypass line via a connecting line;
a connecting line valve coupled to the connecting line; and
a controller configured to: exhaust the connecting line during a substrate process within the reaction chamber.

15. A gas-phase reactor system comprising:
a reactor comprising a reaction chamber;
a gas supply unit coupled to the reaction chamber via a gas supply line;
a first exhaust system coupled to the reaction chamber via a first exhaust line;
a bypass line coupled to the gas supply unit and to the first exhaust system;
a gas detector coupled to the bypass line via a connecting line;
a connecting line valve coupled to the connecting line; and
a controller configured to: exhaust the connecting line by closing the connecting line valve and opening a second exhaust line valve.

16. A gas-phase reactor system comprising:
a reactor comprising a reaction chamber;
a gas supply unit coupled to the reaction chamber via a gas supply line;
a first exhaust system coupled to the reaction chamber via a first exhaust line;
a bypass line coupled to the gas supply unit and to the first exhaust system;
a gas detector coupled to the bypass line via a connecting line;
a connecting line valve coupled to the connecting line; and
a controller configured to: exhaust gas to the first exhaust system and the second exhaust system during a leak check of the gas supply unit.

17. The gas-phase reactor system of claim 16, further comprising a gas inlet line between the gas supply unit and the reactor.

18. The gas-phase reactor system of claim 16, further comprising a gas inlet valve between the gas supply unit and the reactor.
